(19) United States
(12) Patent Application Publication
(10) Pub. No.: US 2005/0209596 A1
(43) Pub. Date: Sep. 22, 2005

(54) Medical instrument

(75) Inventors: Bernd Daniels, Leichlingen (DE); Armin Daniels, Leichlingen (DE)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: FRIEDRICH DANIELS GmbH

(21) Appl. No.: 10/896,564

(22) Filed: Jul. 22, 2004

(30) Foreign Application Priority Data
Mar. 6, 2004 (DE) 102004011103.0

(51) Int. Cl. A61B 17/32

(52) U.S. Cl. 606/83

(57) ABSTRACT

A medical instrument includes a handle having metal cores which are connected with an elongated base or lower member and an elongated upper or movable member. A spring is connected with the metal cores and urges the metal cores toward an initial relationship. Silicone rubber coverings are provided over the metal cores. The silicone rubber covers provide a soft grip which is comfortable for a user of the medical instrument, does not become slippery in an operating environment, and can be cleaned by steam sterilization.

### BACKGROUND OF THE INVENTION

The present invention relates to a new and improved medical instrument, such as a rongeur or punch, which is used in the performance of an operation on a patient's body.

Known rongeurs are formed of metal, such as stainless steel. These known rongeurs have a manually engagable metal handle which can be squeezed against the influence of a biasing spring to effect relative movement between elongated members. When a metal handle is squeezed, one of the elongated members moves relative to the other elongated member to engage bone or other body tissue disposed between end portions of the elongated member.

The metal handles of known rongeurs give a cold feeling and are hard to hold and to handle. In an operating environment, the metal handles of known rongeurs tend to be come slippery. The combination of these factors can result in fatigue of the tactile and manual skills of the user of a known rongeur. Known rongeurs are disclosed in U.S. Pat. Nos. 4,733,663; 5,273,519; 5,961,531; and 6,214,010.

### SUMMARY OF THE INVENTION

The present invention provides a new and improved medical instrument which is used in performing an operation on tissue in a patient's body. The instrument may be a rongeur or a punch. Although the medical instrument may be any one of many known medical instruments, the instrument includes a base member and a second member which is movable relative to the base member. Tissue of a human patient may be disposed between the base and second members. A handle is connected with the base and second members.

The handle includes a first metal core which is connected with the base member. A second metal core is connected with the second member. A spring is connected with the metal cores to urge the metal cores toward an initial spatial relationship.

In accordance with a feature of the present invention, a first cover formed of silicone rubber overlies at least a portion of the first metal core of the handle. A second cover formed of silicone rubber overlies at least a portion of the second metal core of the handle. The first silicone rubber cover is engagable by a palm of a hand of a person using the medical instrument. The second silicone rubber cover is engagable by fingers on the hand of the person using the medical instrument. Manual application of force to the handle effects relative movement between the base and second members against the influence of the spring.

In accordance with another feature of the present invention the spring may be enclosed by a portion of the medical instrument. Alternatively, the spring may by exposed to the environment around the medical instrument.

Although the present invention is believed to be particularly advantageous when utilized in association with a sliding shaft instrument, such as a rongeur or punch, it is contemplated that the present invention may be utilized in association with other known medical instruments. Regardless of what type of medical instrument the invention is used with, comfort of a person using the medical instrument is increased by having the metal cores of the handle covered by silicone rubber. The silicone rubber covers cushion the palm and fingers on a hand of a person using the medical instrument. In addition, the silicone rubber covers reduce slipperiness of the handle of the medical instrument in an operating environment. The silicone rubber coverings on the handle can be cleaned by steam sterilization.

### BRIEF DESCRIPTION OF THE DRAWINGS

The foregoing and other features of the invention will become more apparent upon consideration of the following description taken in connection with the accompanying drawings wherein:

FIG. 1 is a schematic side elevational view of a medical instrument constructed in accordance with the present invention;

FIG. 2 is a sectional view of a portion of the medical instrument of FIG. 1 and illustrating the manner in which a handle is connected with relatively movable members;

FIG. 3 is an enlarged schematic sectional view depicting the manner in which a spring is connected with a portion of the handle;

FIG. 4 is a schematic side elevational view of a second embodiment of a medical instrument constructed in accordance with the present invention, the medical instrument being illustrated in an unactuated condition;

FIG. 5 is an enlarged fragmentary schematic sectional view depicting the construction of a portion of a handle of the medical instrument of FIG. 4;

FIG. 6 is a sectional view of a portion of the unactuated medical instrument of FIG. 4 and depicting the manner in which the handle is connected with relatively movable members;

FIG. 7 is a schematic side elevational view of the medical instrument of FIG. 4 in an actuated condition; and

FIG. 8 is a sectional view of a portion of the actuated medical instrument of FIG. 7 and further depicting the manner in which the handle is connected with the relatively movable members.

### DESCRIPTION OF SPECIFIC PREFERRED EMBODIMENTS OF THE INVENTION

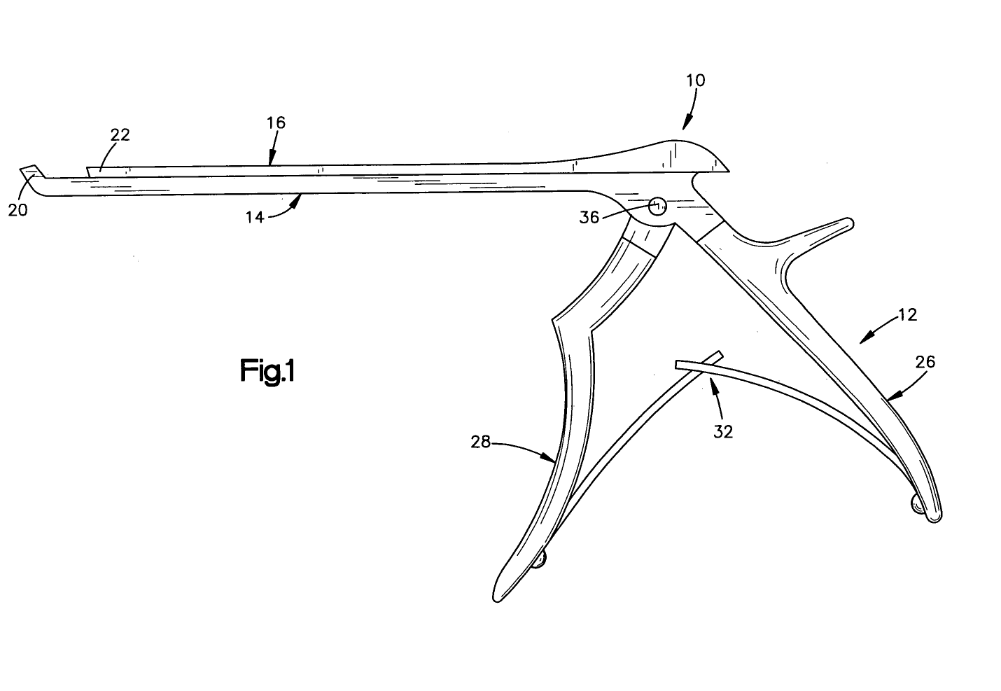

A medical instrument 10 is illustrated schematically in FIG. 1. The medical instrument 10 includes a handle 12 which is connected with a base or lower member 14 and a movable upper member 16. The elongated base or lower member 14 has an end portion 20 which cooperates with an end portion 22 of the movable upper member 16 to engage body tissue upon manual actuation of the handle 12. The lower and upper members 14 and 16 are formed of metal (stainless steel).

The handle 12 includes a base section 26 and a movable section 28. The lower member 14 is fixedly connected to the base section 26 of the handle 12. The upper member 16 is connected to and is movable relative to the movable section 28 of the handle 12.

A spring 32 is disposed between and is connected with the base section 26 and movable section 28 of the handle 12. The spring 32 is exposed to the environment around the medical instrument 10. The spring 32 is effective to urge the base section 26 and movable section 28 of the handle 12 to the initial spatial relationship illustrated in FIG. 1. At this time, the medical instrument 10 is in an unactuated condition with the end portions 20 and 22 of the lower member 14 and upper member 16 spaced apart from each other.

The medical instrument 10 is of the well known sliding shaft type. Thus, upper member 16 is slidable along the lower member 14 against the influence of the spring 32 when the handle 12 is manually actuated. However, it should be understood that the medical instrument 10 may operated in a different manner if desired.

When body tissue is disposed between the end portions 20 and 22 of the lower and upper members 14 and 16, force may be manually applied to the handle 12 to effect movement of the end portion 22 of the upper member 16 toward the end portion 20 of the lower member 14. This relative movement presses the end portion 22 of the upper member 16 against the body tissue disposed between the end portions 20 and 22 of the lower and upper members 14 and 16. The body tissue may be either hard or soft body tissue.

When the handle 12 is to be manually engaged by a surgeon or other individual operating the medical instrument 10, the palm of the users hand engages the base section 26 of the handle 12. The fingers on the users hand engage the movable section 28 of the handle 12. Force transmitted from the users fingers to the movable section 28 of the handle 12 is effective to move the movable section toward the base section 26 of the handle. As this occurs, movable section 28 pivots about a pin 36 as the spring 32 is compressed.

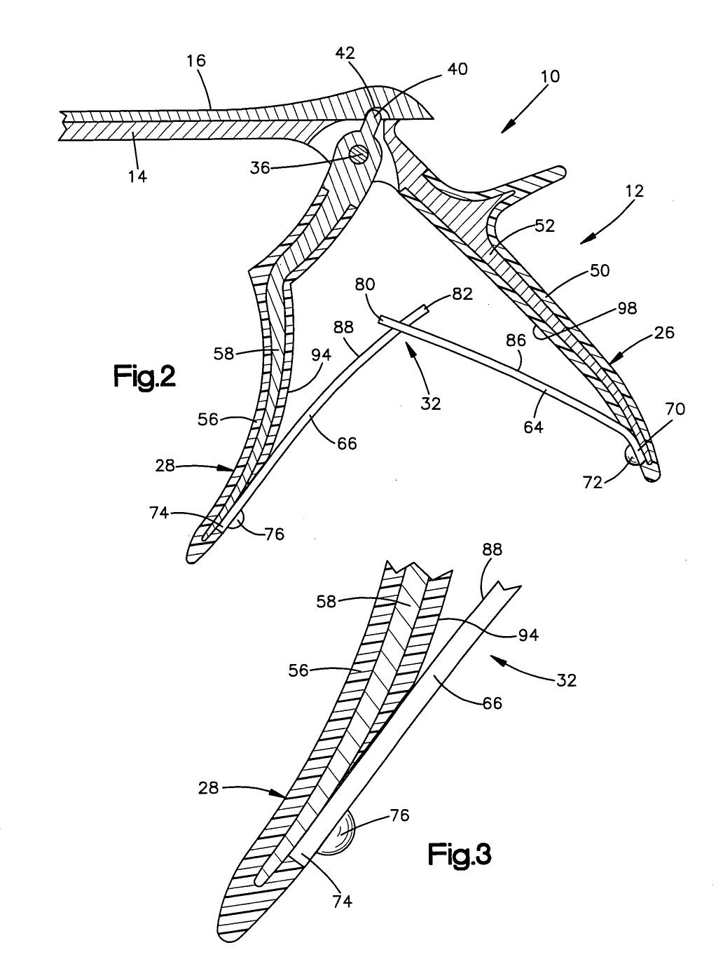

When the movable section 28 is pivoted about the pin 36, a projection 40 (FIG. 2) from the movable section 28 is effective to slide the upper member 16 along the lower member 14. The projection 40 is received in a recess 42 formed in the upper member 16. Therefore, when the movable handle section 28 is pivoted in a counterclockwise direction (as viewed in FIG. 2) about the pin 36, the upper member 16 moves along a linear path toward the left (as viewed in FIGS. 1 and 2). This moves the end portion 22 (FIG. 1) of the upper member 16 toward the stationary end portion 20 of the lower member 14.

The end portion 22 on the movable upper member 16 may be constructed so as to form a sharp blade which cuts body tissue, such as bone, with either an end cutting or side cutting action. Although it is believed that it may be preferred to utilize the medical instrument 10 to remove a small amount of bone, it is contemplated that the medical instrument 10 may be used in association with body tissue other than bone. It is also contemplated that the end portion 22 of the movable upper member 16 may be constructed so as to cooperate with the end portion 20 of the lower member 14 to grip body tissue rather than to cut the body tissue. The illustrated medical instrument 10 is a rongeur. However, the medical instrument 10 may be a punch. The medical instrument 10 may be of a type other than the illustrated sliding shaft type.

Although a specific arrangement for interconnecting the movable section 28 and the upper member 16 is illustrated schematically in FIG. 2, it is contemplated that the upper member 16 and movable section 28 may be connected in a different manner by any one of many suitable connections which have been utilized in association with known rongeurs and/or other medical instruments. Regardless of how the movable section 28 is connected with the upper member 16, movement of the movable section 28 toward the base section 26 of the handle 12 is effective to move the upper member 16 along a linear path which extends parallel to a longitudinal central axis of the lower member 14.

The lower member 14 may be connected with the upper member 16 by a tongue and groove connection. However, any desired connection may be utilized between the lower member 14 and upper member 16. Regardless of what kind of connection is utilized, the upper member 16 moves along the lower member 14 upon movement of the movable section 28 of the handle 12 relative to the base section 26 of the handle. Although the medical instrument 10 is of the sliding shaft type, the medical instrument 10 may have a different type of construction.

In accordance with one of the features of the present invention, the base section 26 of the handle 12 includes a silicone rubber covering 50 (FIG. 2). The silicone rubber covering 50 overlies and is bonded to at least a portion of a rigid metal (stainless steel) core 52 of the base section 26. Similarly, the movable section 28 of the handle 12 includes a silicone rubber covering 56. The silicone rubber covering 56 overlies and is bonded to at least a portion of a rigid metal (stainless steel) core 58 of the movable section 28.

When the movable section 28 is to be moved relative to the base section 26, a surgeon or other person operating the rongeur 10 grips the handle 12. At this time, the palm of the hand of the surgeon is pressed against the relatively soft silicone rubber covering 50 over the rigid metal core 52 on the base section 26. At the same time, the fingers of the person using the medical instrument 10 engage the relatively soft silicone rubber covering 56 over the rigid metal core 58 on the movable handle section 28.

Force which is manually applied against the movable section 28 is effective to pivot the movable section 28 in a counterclockwise direction about the pin 36 and to compress the spring 32. The relatively soft silicone rubber covering 56 on the movable handle section 28 cushions the fingers of the individual using the medical instrument 10 to increase comfort and minimize fatigue. Similarly, the soft silicone rubber covering 50 on the base section 26 of the handle 12 cushions the palm of the hand of the person using the medical instrument 10. This also increases the comfort of the individual using the medical instrument 10 to prevent tiring of the individual during the performance of an operation on a patient's body.

The silicone rubber coverings 50 and 56 on the base section 26 and movable section 28 of the handle 12 have the same composition. The silicone rubber coverings 50 and 56 are an elastomeric material having a Shore A hardness between 75 and 85. The silicone rubber of the coverings 50 and 56 may have a tensile strength of 12 N/mm² and a yield stress of approximately 600 percent. The silicone rubber of the coverings 50 and 56 can be steam sterilized at a temperature of at least 130° Centigrade. To facilitate steam sterilization the silicone rubber of the coverings 50 and 56 can be exposed to temperatures of at least 200° Centigrade without significant deformation. Although the silicone material for the coverings 50 and 56 may be obtained from many different sources, one commercial source of a suitable silicone rubber for the coverings 50 and 56 is Wacker Chemical Corporation of Adrian, Mich.

The rigid metal core 52 is integrally formed as one piece with the lower member 14. The rigid metal core 58 is pivotally connected to the lower member 14 and to the rigid metal core 52 by the pin 36. The rigid metal cores 52 and 58 and lower and upper members 14 and 16 may be formed of any desired material. However, in the embodiment of the invention illustrated in FIG. 2 they are formed of stainless steel.

The spring 32 includes a pair of resiliently deflectable leaf spring members 64 and 66 (FIG. 2). The spring members 64 and 66 extend through the silicone rubber covers 50 and 56 into engagement with the metal (stainless steel) cores 52 and 58. The stainless steel leaf spring member 64 has an end portion 70 which is fixedly connected to the metal (stainless steel) core 52 of the base section 26 of the handle 12 by a fastener 72. Similarly, the stainless steel leaf spring member 66 has an end portion 74 (FIGS. 2 and 3) which is fixedly connected to the metal (stainless steel) core 58 of the movable section 28 of the handle 12 by a fastener 76.

The leaf spring members 64 and 66 have end portions 80 and 82 which are interconnected by a suitable hinge. The hinge may be formed by having one of the end portions 80 or 82 extend through an opening in the other end portion. Alternatively, the hinge may be formed by a pin which is connected with both of the leaf spring members 64 and 66.

When the handle 12 is gripped by a surgeon or other person using the medical instrument 10, force is applied against the base and movable sections 26 and 28 of the handle 12 in the manner previously discussed. This force pivots the movable section 28 about the pin 36 and moves the movable section 28 along the base section 26 against the influence of the spring 32.

As this occurs, the leaf spring members 64 and 66 are resiliently deflected. As the leaf spring members 64 and 66 are resiliently deflected, side surfaces 86 and 88 on the leaf spring members 64 and 66 (FIG. 2) are pressed against the silicone rubber coverings 50 and 56. Thus, as the movable handle section 28 moves toward the base handle section 26, the side surface 88 (FIG. 3) on the leaf spring member 66 is resiliently deflected and pressed against an outer side surface 94 of the covering 56. This enables the covering 56 to provide a soft base against which the leaf spring member 66 is pressed and deflected. The force applied against the silicone rubber covering 56 by the leaf spring member 66 is effective to compress a portion of the silicone rubber covering as the leaf spring member 66 is deflected. This tends to minimize stress concentrations in the leaf spring member 66 and enhances the service life of the medical instrument 10.

As the movable handle section 28 (FIG. 2) moves toward the base handle section 26, the leaf spring member 64 is deflected. As the leaf spring member 64 is deflected, the side surface 86 on the leaf spring member 64 is pressed against an outer side surface 98 on the silicone rubber covering 50. The force applied against the silicone rubber covering 50 by the leaf spring member 64 is effective to compress a portion of the silicone rubber covering 50 and deflect the spring member 64. The covering 50 provides a soft base against which the spring member 64 is pressed and deflected. This tends to minimize stress concentrations in the leaf spring member 64 and to enhance the service life of the medical instrument 10.

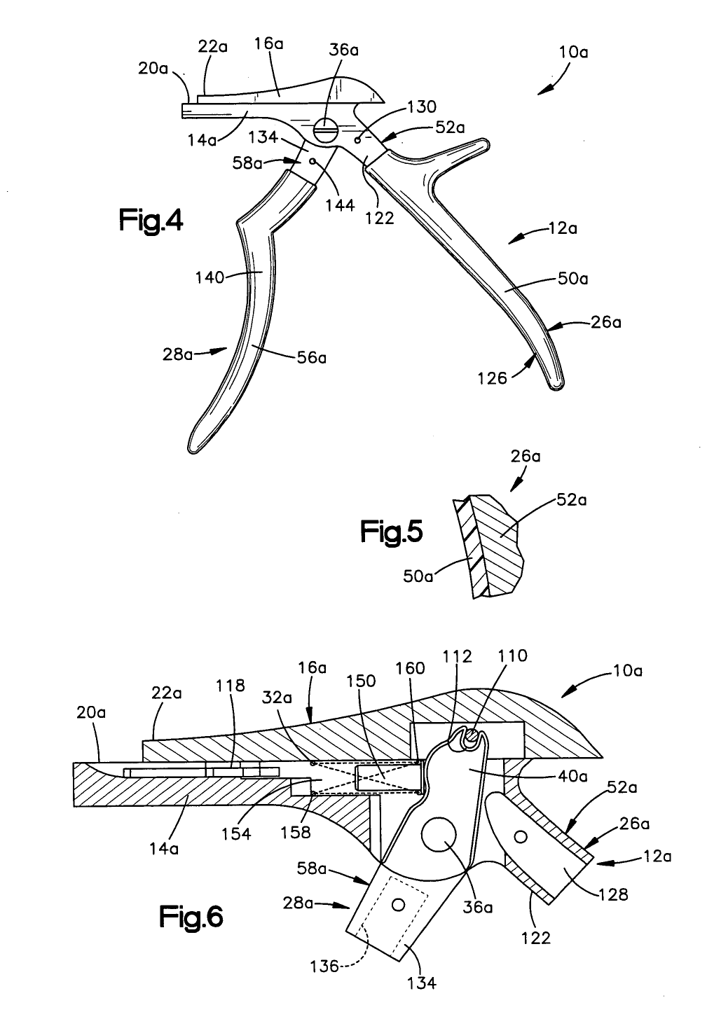

In the embodiment of the medical instrument illustrated in FIGS. 1-3, the spring 32 is directly connected to the handle 12. In the embodiment of the medical instrument illustrated schematically in FIGS. 4-8, the spring is enclosed within the medical instrument and is indirectly connected to the handle of the medical instrument. Since the embodiment of the invention illustrated in FIGS. 4-8 is generally similar to the embodiment of the invention illustrated in FIGS. 1-3, similar numerals will utilized to identify similar components, the suffix letter “a” being added to the numerals of FIGS. 4-8 to avoid confusion.

A medical instrument 10a (FIG. 4) includes a handle 12a which is connected with a base or lower member 14a and a movable upper member 16a. The base or lower member 14a has an end portion 20a which cooperates with an end portion 22a of the movable upper member 16a to engage body tissue upon manual actuation of the handle 12a. The upper and lower members 14a and 16a are formed of metal (stainless steel).

The handle 12a includes a base section 26a (FIG. 4) and a movable section 28a. The lower member 14a is fixedly connected to the base section 26a of the handle 12a. The upper member 16a is connected to and is movable relative to the movable section 28a of the handle 12a.

In accordance with a feature of this embodiment of the invention, a spring 32a (FIG. 6) is enclosed by other components of the medical instrument 10a. By having the spring 32a enclosed by other components of the medical instrument 10a, the medical instrument is easier to clean and there is less risk of injury.

The spring 32a is connected with the handle 12a. The spring 32a is effective to urge the base section 26a and movable section 28a of the handle 12a to the initial spatial relationship illustrated in FIG. 4. At this time, the end portions 20a and 22a of the lower member 14a and upper member 16a are spaced apart from each other.

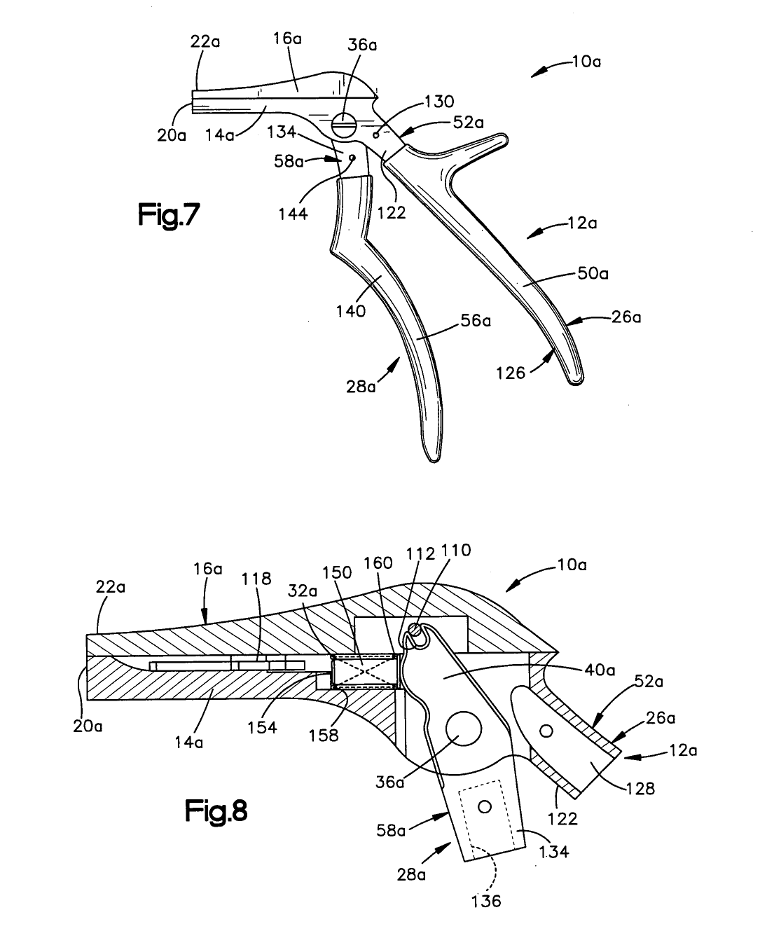

The medical instrument 10a is of the well known sliding shaft type. Thus, the upper member 16a is slidable along the lower member 14a against the influence of the spring 32a when the handle 12a is manually actuated. Manual actuation of the handle 12a causes the upper member 16a to move from the initial position illustrated in FIGS. 4 and 6 to the actuated position illustrated in FIGS. 7 and 8. It should be understood that the medical instrument 10a may operate in a different manner than that illustrated in FIGS. 4-8 if desired.

When the handle 12a is to be manually engaged by a surgeon or other individual operating the medical instrument 10a, the palm of the users hand engages the base section 26a (FIG. 4) of the handle 12a. The fingers on the users hand engage the movable section 28a of the handle 12a. Force transmitted from the users fingers to the movable section 28a of the handle 12a is effective to move the movable section toward the base section 26a of the handle. As this occurs, the movable section 28a pivots about a pin 36a as the spring 32a (FIGS. 6 and 8) is compressed.

When the movable section 28a is pivoted about the pin 36a, a projection 40a (FIG. 6) from the movable member 28a is effective to slide the upper member 16a along the lower member 14a. A pin 110 on the upper member 16a extends into a slot 112 in the projection 40a on the movable section 28a. Therefore, when the movable handle section 28a is pivoted in a counterclockwise direction (as viewed in FIG. 6) about the pin 36a, the upper member 16a slides along a linear path towards the left from the initial position illustrated in FIGS. 4 and 6 to the actuated position illustrated in FIGS. 7 and 8. This results in movement of the end portion 22a (FIGS. 4 and 6) of the upper member 16a toward the stationary end portion 20a of the lower member 14a.

The end portion 22a of the upper movable member 16a may be constructed to form a sharp blade or edge which cuts body tissue, such as bone or soft tissue, with a side cutting action. Although it is believed it may be preferred to utilized the medical instrument 10a to remove a small amount of bone, it is contemplated that the medical instrument 10a may be used in association with body tissue other than bone. It is also contemplated that the end portion 22a of the movable upper member 16a may be constructed as so to cooperate with the end portion 20a of the lower member 14a to grip body tissue rather than to cut the body tissue. The illustrated medical instrument 10a is a rongeur. However, the medical instrument 10a may be a punch. The medical instrument 10a may be of a type other than the illustrated sliding shaft type.

Although a specific arrangement for interconnecting the movable handle section 28a and upper member 16a is illustrated schematically in FIGS. 6 and 8, it is contemplated that the upper member 16a and movable handle section 28a may be interconnected in a different manner by any one of many suitable connections which have been utilized in association with rongeurs and/or other medical instruments. Regardless of how the movable handle section 28a is connected with the upper member 16a, movement of the movable section 28a toward the base section 26a of the handle 12a is effective to move the upper member 16a along a linear path which extends parallel to a longitudinal central axis of the lower member 14a.

The lower member 14a is connected the upper member 16a by a tongue and groove connection 118 (FIGS. 6 and 8). However, any desired connection may be utilized between the lower member 14a and the upper member 16a. Regardless of what kind of connection is utilized, the upper member 16a moves along the lower member 14a upon movement of the movable section 28a of the handle 12a relative to the base section 26a of the handle. Although the medical instrument 10a is of the sliding shaft type, the medical instrument 10a may have a different type of construction.

In accordance with one of the features of the present invention, the base section 26a of the handle 12a includes a silicone rubber covering 50a (FIGS. 4 and 5). The silicone rubber covering 50a overlies and is bonded to at least a portion of a rigid metal (stainless steel) core 52a of the base section 26a. Similarly, the moveable section 28a of the handle 12a includes a silicone rubber covering 56a. The silicone rubber covering 56a overlies and is bonded to at least a portion of a rigid metal (stainless steel) core 58a of the movable section 28a.

The base section 26a of the handle 12a is formed as two separate parts. A first or upper part 122 (FIGS. 4 and 6) is integrally formed as one piece of metal with the base or lower member 14a. The first or upper part 122 of the handle base section 26a is not covered by the silicone rubber covering 50a. However, the first or upper part 122 of the handle base section 26a may be covered by the silicone rubber covering 50a if desired.

A lower part 126 (FIGS. 4 and 7) of the base section 26a has a cylindrical metal projecting portion (not shown) which extends into a cylindrical socket 128 (FIG. 6) formed in the upper part 122 of the metal core 52a. A pin 130 (FIG. 4) extends through the upper and lower parts 122 and 126 of the base section 126 of the handle 12a to fixedly interconnect the upper and lower parts of the base section. The lower part 126 of the base section 26a includes a metal core which is enclosed by the silicone rubber covering 50a. However, the cylinderical metal projecting portion, which is telescopically received in the socket 128, is not covered by the silicone rubber covering 50a.

The core 58a of the movable section 28a is formed of two parts, in the same general manner as previously described in conjunction with the base section 26a. The core 58a includes an upper part 134 (FIGS. 4 and 6) which includes the projection 40a. The upper part 134 is integrally formed as one piece of metal (stainless steel) and includes a cylindrical socket 136. The upper part 134 of the movable handle section 28a is not covered by the silicone rubber covering 56a. Therefore the projection 40a is not covered by the silicone rubber covering 56a.

A lower part 140 of the movable handle section 28a includes a metal core having a cylindrical projecting portion which extends into the socket 136 and is held in place by a pin 144 (FIGS. 4 and 7). The covering 56a is disposed on the portion of the core 58a which is in the lower part 140 of the moveable section 28a. However, the cylinderical projecting portion, which is telescopically received in the socket 136, is not covered by the silicone rubber covering 56a.

When the movable section 28a is to be moved relative to the base section 26a, a surgeon or other person operating the medical instrument 10a grips the handle 12a (FIG. 4). At this time, the palm of the hand of the surgeon is pressed against the relatively soft silicone rubber covering 50a (FIG. 5) over the rigid metal core 52a on the base section 26a. At the same time, the fingers of the person using the medical instrument 10a (FIG. 4) engage the relatively soft silicone rubber covering 56a over the rigid metal core 58a on the movable handle section 28a.

Force which is manually applied against the movable handle section 28a is effective to pivot the movable section in a counterclockwise direction about the pin 36a. This results in the projection 40a (FIG. 6) applying force to a metal plunger 150 which is disposed in a coaxial relationship with the helical coil spring 32a. The force transmitted from the projection 40a is effective to compress the spring 32a (FIG. 8). The plunger 150 engages the projecting portion 40a of the core 58a at a location spaced from the covering 56a.

The relatively soft silicone rubber covering 56a on the moveable handle section 28a cushions the fingers of the individual using the medical instrument 10a to increase comfort and minimize fatigue. Similarly, the soft silicone rubber covering 50a on the base section 26a of the handle 12a cushions the palm of the hand using the medical instrument 10a. This also increases the comfort of the individual using the medical instrument 10a to prevent tiring of the individual during the performance of an operation on a patient's body.

The silicone rubber coverings 50a and 56a on the base section 26a and movable section 28a of the handle 12a have the same composition. The silicone rubber coverings 50a and 56a on the base section 26a and movable section 28a of the handle 12a have the same composition and same characteristics as previously described in conjunction with the silicone rubber coverings 50 and 56 of FIGS. 1-3.

The spring 32a is enclosed within the medical instrument 10a. Thus, the spring 32a is received in a slot or recess 154 in the base or lower member 14a. The slot 154 has a generally U-shaped cross sectional configuration as viewed in a plane extending perpendicular to parallel longitudinal central axes of the lower and upper members 14a and 16a. The upper member 16a extends across the upper end of the slot or recess 154 to close the slot.

The spring 32a is a cylinderical helical coil spring. The first end portion 158 of the spring 32a abuts an end portion of the slot or recess 154. The opposite end portion 160 of the spring abuts an annular head end portion of the plunger 150. The slot or recess 154 has an arcuate bottom surface with a radius of curvature which is slightly greater than the outside radius of curvature of the cylinderical spring 32a. The center of curvature of the arcuate bottom surface of the slot or recess 154 is aligned with the central axis of the spring 32a. The slot or recess 154 has parallel side surfaces which extend upward (as viewed in FIG. 6) from the bottom of the slot toward the upper member 16a

When the handle 12a is in the initial condition illustrated in FIGS. 4 and 6, the plunger 150 transmits force from the spring 32a to the projection 40a to maintain the movable section 28a of the handle 12a in the unactuated position illustrated in FIGS. 4 and 6. Upon application of manual force to the movable section 28a of the handle 12a, the movable handle section is pivoted in a counterclockwise direction (as viewed in FIG. 4) about the pin 36a. As this occurs, the upper member 16a slides along the lower member 14a to move the end portion 22a of the upper member 16a toward the end portion 20a of the lower member 14a. As this occurs, the plunger 150 is moved toward the left (as viewed in FIG. 6) to compress the spring 32a (FIG. 8).

In view of the foregoing description, it is apparent the present invention provides a new and improved medical instrument 10, 10a which is used in performing an operation on tissue in a patient's body. Although the medical instrument 10, 10a may be any one of many known medical instruments, the instrument is a rongeur. The instrument 10, 10a includes a base member 14, 14a and a second member 16, 16a which is movable relative to the base member with tissue disposed between the base and second members. A handle 12, 12a is connected with the base and second members 14, 14a and 16, 16a.

The handle 12, 12a includes a first metal core 52, 52a which is connected with the base member 14, 14a. A second metal core 58, 58a is connected with the second member 16, 16a. A spring 32, 32a is connected with the metal cores 52, 52a and 58, 58a to urge the metal cores toward an initial spatial relationship.

In accordance with a feature of the present invention, a first cover 50, 50a formed of silicone rubber overlies at least a portion of the first metal core 52, 52a of the handle 12, 12a. A second cover 56, 56a formed of silicone rubber overlies at least a portion of the second metal core 58, 58a of the handle 12, 12a. The first silicone rubber cover 50, 50a is engagable by a palm of a hand of a person using the medical instrument 10, 10a. The second silicone rubber cover 56, 56a is engagable by fingers on the hand of the person using the medical instrument 10, 10a. Manual application of force to the handle 12, 12a effects relative movement between the base and second members 14, 14a and 16, 16a against the influence of the spring 32, 32a.

Although the present invention is believed to be particularly advantageous when utilized with a sliding shaft type of instrument, it is contemplated that the present invention may be utilized in association with other known medical instruments. Regardless of what type of medical instrument the invention is utilized with, comfort of the person using the medical instrument is increased by having the metal cores 52, 52a and 58, 58a of the handle 12, 12a covered by silicone rubber. The silicone rubber covers 52, 52a and 58, 58a cushion the palm and fingers on a hand of a person using the medical instrument 10. In addition, the silicone rubber covers 52, 52a and 58, 58a reduce slipperiness of the handle 12, 12a of the medical instrument 10, 10a in an operating environment. The silicone rubber coverings 50, 50a and 56, 56a on the handle 12, 12a can be cleaned by steam sterilization.

1. A medical instrument for use in performing an operation on tissue in a patient's body, said instrument comprising a first member, a second member which is movable relative to the first member with tissue disposed between portions of the first and second members, and a handle connected with said first and second members, said handle includes a first metal core connected with said first member, a second metal core connected with said second member, a first cover formed of silicone rubber and overlying at least a portion of said first metal core, a second cover formed of silicone rubber and overlying at least a portion of said second metal core, and a spring connected with said handle to urge said first and second metal cores toward an initial spatial relationship, said first cover being engagable by a palm on a hand of a person using said instrument and said second cover being engagable by fingers on the hand of the person using said instrument to enable force to be manually applied to said handle to effect relative movement between said first and second members against the influence of said spring.

2. A medical instrument as set forth in claim 1 wherein said second member is movable along said first member under the influence of force which is applied to said second cover by fingers on the hand of the person using said instrument, said second member having an end portion which moves toward an end portion of said first member to enable body tissue to be engaged between said end portions of said first and second members.

3. A medical instrument as set forth in claim 1 wherein said first member is fixedly connected with said first metal core, said second metal core being pivotal relative to said first metal core to move said second member along said first member.

4. A medical instrument as set forth in claim 1 wherein said spring includes a resiliently deflectable spring member having an end portion which is connected to one of said first and second metal cores, a portion of said spring member being pressed against one of said first and second covers upon the application of manual force to said handle to effect relative movement between said first and second members.

5. A medical instrument as set forth in claim 1 wherein said spring is at least partially disposed in a recess which is at least partially formed in said first member and provides a biasing force which is transmitted to said second core at a location spaced from said second cover.

6. A medical instrument as set forth in claim 1 wherein said first and second covers have shore A hardness of 75 to 85.

7. A medical instrument as set forth in claim 1 wherein said first and second covers can be heated to a temperature of at least 130 degrees Centigrade without permanent deformation of said first and second covers.

8. A medical instrument as set forth in claim 1 wherein a first end portion of said spring extends through said first cover and is connected to said first metal core, and a second end portion of said spring extends through said second cover and is connected to said second metal core.

9. A medical instrument for use in performing an operation on tissue in a patient's body, said instrument comprising a first member, a second member which is movable relative to the first member with tissue disposed between portions of the first and second members, and a handle connected with said first and second members, said handle includes a first section connected with said first member, a second section connected with said second member, and a spring connected with said handle to urge said first and second section toward an initial spatial relationship, said spring is at least partially disposed in a recess which is formed in said first member and provides a biasing force which is transmitted to said second section, said first section being engagable by a palm on a hand of a person using said instrument and said second section being engagable by fingers on the hand of the person using said instrument to enable force to be manually applied to said handle to effect relative movement between said first and second members against the influence of said spring.

10. A medical instrument as set forth in claim 9 wherein said second member is movable along said first member under the influence of force which is applied to said second section by fingers on the hand of the person using said instrument, said second member having an end portion which moves toward an end portion of said first member to enable body tissue to be engaged between said end portions of said first and second members.

11. A medical instrument as set forth in claim 9 wherein said spring is a helical coil spring, the recess in the first member having an arcuate side surface with a center of curvature which is aligned with a central axis of said helical coil spring.

12. A medical instrument as set forth in claim 9 wherein said spring is a helical coil spring, said instrument further includes a plunger disposed in a coaxial relationship with said spring to transmit force from said spring to urge said first and second sections toward the initial spatial relationship.

13. A medical instrument as set forth in claim 12 wherein said first section of said handle includes a first metal core which is at least partially enclosed by a first cover formed by silicone rubber, said first cover being engagable by a palm of on a hand of a person using said instrument, said second section of said handle includes a second metal core which is at least partially enclosed by a second cover formed of silicone rubber, said second cover being engagable by the fingers on the hand of a person using said instrument.

14. A medical instrument as set forth in claim 13 wherein an end portion of said plunger is disposed in engagement with said second metal core at a location spaced from said second cover.
